(19) United States
(12) Patent Application Publication
(10) Pub. No.: US 2025/0136922 A1
(43) Pub. Date: May 1, 2025

(54) SYSTEM AND METHOD FOR CULTIVATING AND HARVESTING CYANOBACTERIAL BIOMASS

(71) Applicant: BOTANICO DESIGN LTD, London (GB)

(72) Inventors: Robert Russell, London (GB); Edward Laney, London (GB); Umar Saleem, London (GB)

(21) Appl. No.: 18/683,440

(22) PCT Filed: Aug. 12, 2022
(86) PCT No.: PCT/IB2022/057547

§ 371 Date: Feb. 13, 2024

(30) Foreign Application Priority Data
Aug. 13, 2021 (GB) 2111650.4

(51) Int. Cl. C12N 1/20; C12M 1/00; C12M 1/34; C12M 3/00

(52) U.S. Cl. CPC C12N 1/20; C12M 21/02; C12M 23/44; C12M 29/08; C12M 31/10; C12M 41/06; C12M 41/30

(57) ABSTRACT

Disclosed is a system for cultivating and harvesting a cyanobacterial biomass. The system comprises at least one vessel that, when in operation, grows the cyanobacterial biomass in a nutrient growth media under regulated growth conditions and a base unit configured to receive the at least one vessel. The system also comprises at least one light source and a cultivation air pump that, when in operation, supplies light, heat and air to the at least one vessel. Notably, the cultivation air pump supplied air via a one-way air valve in the at least one vessel. The system also comprises at least one photodiode that, when in operation, measure a cyanobacterial biomass concentration in the at least one vessel. Disclosed also is a method for cultivating and harvesting a cyanobacterial biomass.

### TECHNICAL FIELD

The present disclosure relates generally to cell cultivation; and more specifically, to systems for cultivating and harvesting cyanobacterial biomass. The present disclosure also relates to methods for cultivating and harvesting cyanobacterial biomass using the aforementioned systems.

### BACKGROUND

In recent times, people have become increasingly health conscious, and therefore, are transitioning towards a healthy diet. It will be appreciated that a healthy diet helps to prevent many chronic non-communicable diseases, such as heart disease, diabetes, and cancer, and further helps to improve a person's overall physical and mental health. Therefore, people include nutrition (or dietary) supplements in addition to healthy food in their diet. Such nutrition (or dietary) supplements could be provided by microalgae, such as cyanobacteria, red algae, seaweed, and the like, that have a high concentration of vitamins, minerals and other essential nutrients. In this regard, demand for microalgae species, for example spirulina, has rapidly increased in recent times. Notably, the global presence of such food sources, including cyanobacteria, especially spirulina, is very limited and fails to sufficiently meet the nutrient demand of the consumers worldwide. Therefore, such food sources are commercially produced, for example, using large-scale or lab-scale open-air cultivation techniques, closed bioreactors, and so forth.

Typically, open-air cultivation is performed in a natural environment of the cyanobacteria. However, the cyanobacteria, such as spirulina, could be contaminated in such an environment, for example with microcystins. Such contamination may occur as a result of mixing of spirulina batches with other, cyanotoxin-producing blue-green algae. Furthermore, spirulina may potentially be contaminated with heavy metals, such as lead, mercury, and arsenic. Therefore, there is a need for a means, such as bioreactors, for cultivating spirulina that is safe for human consumption.

Conventionally, systems such as bioreactors provide a suitable artificial environment, through accurate measurement and control of inputs and outputs, such as carbon dioxide concentration, nutrient growth media formulation, light level, temperature, and so on, to produce a cyanobacterial biomass. However, the production in such systems is costly due to production of the cyanobacterial biomass in small batches therein, thus increasing the overall cost of the end product. Additionally, such a production process is inherently complex as it is designed for professionals, such as scientists, to use, thus making it difficult for consumers lacking any scientific knowledge or expertise to operate such systems. Moreover, the size of such systems is often big enough to require a dedicated space for installation thereof, thereby limiting its use as a regular home appliance.

Recent advances in the field of spirulina cultivation have introduced solutions for providing fresh spirulina at cheaper prices. In this regard, compact bioreactors, namely consumer photobioreactors (PBRs), have been designed in order to address drawbacks associated with the conventional bioreactors. Typically, the consumer PBRs are employed to grow, at home, a small amount of spirulina to meet a daily requirement thereof. However, existing consumer PBRs require frequent human intervention, such as, for example, to estimate when the spirulina is ready to harvest, thus are not user-friendly. Moreover, the harvesting step in the conventional consumer PBRs is time consuming as it is based on, for example, gravity or percolation techniques. Furthermore, the harvested spirulina is often required to undergo downstream processing (such as rinsing, drying, and the like), thereby increasing the overall cost of the end product. Additionally, the spirulina also loses some of its nutritive ingredients or other desired qualities during the downstream processing, thereby defeating the purpose of fresh cultivation.

Therefore, considering the foregoing discussion, there exists a need to overcome drawbacks associated with conventional techniques for cultivating and harvesting a cyanobacterial biomass.

### SUMMARY

The present disclosure seeks to provide a system for cultivating and harvesting a cyanobacterial biomass. The present disclosure also seeks to provide a method for cultivating and harvesting a cyanobacterial biomass. The present disclosure seeks to provide a solution to the existing problem of cultivation and harvesting of fresh biomass. An aim of the present disclosure is to provide a solution that overcomes at least partially the problems encountered in prior art, and provides an efficient, reliable, eco-friendly, user-friendly, and cost-efficient system.

In one aspect, an embodiment of the present disclosure provides a system for cultivating and harvesting a cyanobacterial biomass, the system comprising:
at least one vessel that, when in operation, grows the cyanobacterial biomass in a nutrient growth media under regulated growth conditions, the at least one vessel having a top end, a bottom end, at least one side wall connecting the top end to the bottom end, and a one-way air valve at the bottom end;
at least one light source that, when in operation, supplies light and heat to the at least one vessel, wherein the at least one light source is arranged on a side of the at least one vessel;
at least one photodiode that, when in operation, measures a cyanobacterial biomass concentration in the at least one vessel, wherein the at least one photodiode is arranged opposite to the at least one light source;
a base unit configured to receive the bottom end of the at least one vessel; and
a cultivation air pump that, when in operation, supplies air to the at least one vessel via the one-way air valve therein.

In another aspect, an embodiment of the present disclosure provides a method for (of) cultivating and harvesting a cyanobacterial biomass by using the aforementioned system, the method comprising:
receiving, on the base unit, the at least one vessel containing the cyanobacterial biomass and the nutrient growth media;
powering up at least one light source, and a cultivation air pump for supplying the light and air respectively, to the at least one vessel;
growing the cyanobacterial biomass in the at least one vessel under regulated growth conditions;
measuring, using the at least one photodiode, the cyanobacterial biomass concentration in the at least one vessel; and
indicating to the user, via the microcontrollers, that the cyanobacterial biomass is ready to harvest, when the cyanobacterial biomass concentration reaches the pre-defined value.

In yet another aspect, an embodiment of the present disclosure provides an application programming interface executable on a computing device associated with a user of the aforementioned system, wherein the application programming interface is communicably coupled with the microcontrollers, and wherein the application programming interface is configured to provide a signal to the microcontrollers, for setting growth parameters that are used for regulating operation of the at least one light source, the at least one photodiode, the electrical circuitry, and the cultivation air pump.

In still another aspect, an embodiment of the present disclosure provides a composition comprising a cyanobacterial biomass as produced by the aforementioned system or obtained by performing the aforementioned method.

Embodiments of the present disclosure substantially eliminate or at least partially address the aforementioned problems in the prior art, and enable efficient cultivation and harvesting of cyanobacterial biomass, such as spirulina. In this regard, the cultivation and harvesting are performed using a consumer photobioreactor (PBR), in particular one that is portable, compact, and cost-efficient, unlike the conventional photobioreactors. Beneficially, the PBR supplies a fresh amount of spirulina for daily consumption by the consumer as a whole food or a food ingredient. Additionally, beneficially, the PBR reduces frequent human intervention required to ascertain the time for harvesting the biomass based on a growth thereof.

Additional aspects, advantages, features and objects of the present disclosure would be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those skilled in the art will understand that the drawings are not to scale. Wherever possible, like elements have been indicated by identical numbers.

Embodiments of the present disclosure will now be described, by way of example only, with reference to the following diagrams wherein:

FIG. 1 is a system for cultivating and harvesting a cyanobacterial biomass, in accordance with an embodiment of the present disclosure;

FIG. 2. is a perspective view of the system for cultivating and harvesting a cyanobacterial biomass, in accordance with an embodiment of the present disclosure;

FIG. 3 is a cross-sectional view of the base unit and a lower part of the vessel, in accordance with an embodiment of the present disclosure;

FIGS. 4A and 4B are schematic illustrations of the harvesting arrangement for harvesting cyanobacterial biomass, in accordance with an embodiment of the present disclosure;

FIGS. 5A and 5B are schematic illustrations of different stages of a density detection system, in accordance with an embodiment of the present disclosure; and

FIG. 6 is a flowchart of steps of a method for (of) cultivating and harvesting a cyanobacterial biomass by using the aforementioned system, in accordance with an embodiment of the present disclosure.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item at which the arrow is pointing.

### DETAILED DESCRIPTION OF EMBODIMENTS

The following detailed description illustrates embodiments of the present disclosure and ways in which they can be implemented. Although some modes of carrying out the present disclosure have been disclosed, those skilled in the art would recognize that other embodiments for carrying out or practising the present disclosure are also possible.

The present disclosure provides the aforementioned system for cultivating and harvesting the cyanobacterial biomass. The system employs various components that are operatively coupled to form an advanced photobioreactor (PBR) with customizable (namely, regulatable) growth conditions. Moreover, the system is a compact system that requires minimal effort and no prior scientific knowledge for operating it. Moreover, the system comprises portable vessels having a one-way air valve that provides air and liquid sealing mechanisms when the vessels are not in a docking position. The vessel is operable to grow and harvest preferably 0.5-1 g of fresh cyanobacterial biomass, such as spirulina, in 1 day. Additionally, the system removes unnecessary, high-cost features from conventional PBRs, such as nutrient growth media dosing, carbon dioxide delivery, and frequent and trained manual intervention, thereby reducing cost and increasing user friendliness. Accordingly, the system of the present disclosure may be constructed not to include a dosing of the nutrient growth media. The system of the disclosure may be constructed not to include a dedicated carbon dioxide delivery. Moreover, the system allows regulation of the growth conditions to be automated, thereby enabling users with no prior experience to cultivate fresh spirulina at home. Furthermore, the system enables convenient harvesting of the cyanobacterial biomass, such as in a kitchen sink, making this functionality valuable for consumer use. Additionally, the arrangement of light sources, at least one photodiode, and other sensors enable real-time growth tracking of the cyanobacterial biomass in the vessel. Beneficially, the at least one photodiode measures the light intensity incident thereupon to measure the corresponding biomass concentration and indicate to a user, via an associated indication means, that the cyanobacterial biomass is ready for harvesting.

Pursuant to the embodiments of the present disclosure, disclosed is the system and method for cultivating and harvesting the cyanobacterial biomass. Throughout the present disclosure, the term “cultivating” (or “cultivate” or “cultivation”) as used herein refers to a process of growing cells (or microorganisms), having a growth rate, from a small number (or size) to a larger number (or size), in an artificially created environment under regulated growth conditions. Said regulated growth conditions are suitable for an optimal growth of the cells (or microorganisms) in a suitable vessel for growth, nutrient growth media (comprising nutrient feed, growth factors, hormones, salts), and physicochemical parameters (such as relative gases (carbon dioxide, oxygen, methane, and so forth) concentration, pH, osmotic pressure, humidity, temperature, sterile conditions). Moreover, an inoculum comprising a small number of cells (or microorganisms) is provided as an input to the system and a larger number of cells (or microorganisms) are received (namely, harvested) as an output. It will be appreciated that the larger number of cells (or microorganisms) gives a measure of the mass of the living component, namely the biomass, in the vessel. Moreover, the biomass will be different, in terms of for example density thereof, at different stages of growth.

The term “harvesting” (or “harvest” or “harvested”) as used herein refers to a removal of the grown biomass, having desired density, from the vessel. The harvested biomass is suitably processed, using one or more downstream processes, for human consumption as a food, a food ingredient, a nutraceutical, a dietary supplement, a cosmetic ingredient, and so forth. Optionally, the harvested biomass could be used as an additive feed supplement for poultry, water animals, and so forth. It will be appreciated that fresh inoculum may be added to the system after harvesting a previously grown biomass.

Throughout the present disclosure, the term “cyanobacterial biomass” as used herein refers to a biomass of cyanobacteria (also called blue-green algae, a prokaryotic algae). Cyanobacteria is a phylum comprising photosynthetic bacteria that live in aquatic habitats and moist soils. The cyanobacteria typically have pigments (such as phycobilisomes, embedded in intracytoplasmic membranes thereof, chlorophyll, and so forth) that enable the cyanobacteria to synthesize their own food through photosynthesis. Notably, some cyanobacteria may lack phycobilisomes and have alternate pigments (such as chlorophyll b) therein to enable photosynthesis therein. However, some cyanobacteria may have both chlorophyll as well as phycobilisomes. Most cyanobacteria are obligate phototrophs (grow in presence of light); however, some can grow in dark conditions when provided with sufficient supply carbon and energy sources. It will be appreciated that cyanobacteria play an important role in producing gaseous oxygen as a by-product of photosynthesis. Beneficially, the cultivation of cyanobacterial biomass also contributes to increasing oxygen concentration in the atmosphere and oceans (namely, Great Oxygenation Event (GOE)). Moreover, some cyanobacteria are capable of nitrogen fixation. Cyanobacteria typically reproduce at explosive rates, forming dense cyanobacterial biomass concentrations known as blooms. Notably, the cyanobacterial blooms can colour a dispersion media thereof, such as a body of water in its natural environment or the nutrient growth media for growing cyanobacteria in an artificial environment. In such case, the dispersion media takes on a shade of a given cyanobacteria (such as green, red, and so forth depending on the pigment therein) that changes from a translucent shade to opaque shade with increasing density of the cyanobacterial biomass therein. It will be appreciated that besides the cyanobacterial biomass, the disclosed system could be used to grow other organisms by regulating the growth conditions suitable therefor. Optionally, the other organisms include other plant-like protists, microalgae, macroalgae, purple bacteria, plants, mosses, and the like.

Optionally, the cyanobacteria biomass is spirulina. Spirulina is a phototrophic, filamentous and multicellular cyanobacteria that grows in fresh water as well as salt water. The spirulina includes three species Arthrospira platensis, Arthrospira maxima and Arthrospira fusiformis. Moreover, spirulina could be consumed by humans (in fresh form or in the form of tablet or powder for example) and animals (feed supplement). A dried biomass of spirulina typically comprises about 50-75% by weight protein, and the 25-50% by weight water, carbohydrates, fat, antioxidant pigments (phycobiliproteins and carotenoids) and other essential nutrients, such as vitamins (for example, thiamine, riboflavin, niacin, and so on) and minerals (for example, iron, manganese, and so on). Additionally, spirulina has antioxidant, pain-relief, anti-inflammatory and brain-protective properties. It will be appreciated that in its natural environment, spirulina could be contaminated with toxins and/or heavy metals, as discussed above. Optionally, the toxins include neurotoxins, hepatotoxins, cytotoxins, and endotoxins, which can cause gastrointestinal upset, headache, muscle pain, facial flushing, sweating, and in some cased liver damage, and respiratory failure in humans and/or animals that ingest them through contaminated water. Therefore, there is a need for means for cultivating spirulina that is safe for human consumption.

In this regard, the system as disclosed is implemented as a photobioreactor (PBR), such as a consumer photobioreactor, configured for cultivating and harvesting small amounts of cyanobacterial biomass. The term “photobioreactor” of “PBR” typically as used herein refers to a closed system that utilises a light source to cultivate phototrophic organisms, such as the cyanobacteria. It will be appreciated that the PBR provides an artificial environment that carefully regulates and controls specific growth conditions specific for respective organisms. Moreover, the PBR allows much higher growth rates and purity levels (due to a minimal risk of contamination) than the natural environments of the respective organisms. Optionally, the PBR could have a different number of operable units, and each of said units having different shapes, forms, volumes, fabrication materials, and so forth. It will be appreciated that the PBR could be scaled up or down as required by the consumer. In this regard, the PBR could be designed such as to cater to a daily need of the cyanobacterial biomass of a single person or a group of people.

The system comprises at least one vessel that, when in operation, grows the cyanobacterial biomass in nutrient growth media under regulated growth conditions. The at least one vessel is intended to provide a space for biological and/or biochemical reactions required for growing an inoculum of micro-organisms (cyanobacteria) and production of biomass under regulated growth conditions (namely, defined and controlled physical and chemical conditions) for consumption by humans (or animals). Said regulated growth conditions are suitable for an optimal growth of cyanobacterial biomass in nutrient growth media comprising nutrient feed, growth factors, hormones, and so forth. Optionally, the nutrient growth media is a solid media that is dissolved in water (such as distilled water or double distilled water). Optionally, the nutrient growth media comprises at least one of: a source of phosphorus (such as potassium hydrogen phosphate, single super phosphate), a source of sodium (such as sodium bicarbonate, sodium chloride, crude sea-salt (Syahat salt)), a source of potassium (such as Muriate of potash), a source of nitrogen (such as ammonium nitrate, ammonium nitrate, urea), and so on. Optionally, the nutrient growth media could be any of the commercially available standard culture medium, such as Zarrouk's medium, a standard media, a modified media, sea water fortified with various nutrients or fertilizers, and so forth. Optionally, 6000-12000 mg of nutrient growth media is dissolved in a litre of water to result in a dissolved nutrient growth media solution. Subsequently, an inoculum of cyanobacterial biomass is added to the dissolved nutrient growth media solution, to achieve a cyanobacterial biomass density in a range of 100-1000 mg/L. It will be appreciated that the biomass is grown in the at least one vessel using processes adhering to good manufacturing practices under good manufacturing practice (GMP) conditions.

The at least one vessel having a top end, a bottom end, at least one side wall connecting the top end to the bottom end, and a one-way air valve at the bottom end. The at least one vessel contains the nutrient growth media and the inoculum of cyanobacteria. Furthermore, the at least one vessel has the top end, the bottom end, a height defined by the distance between the top end and the bottom end, and a diameter (or width) defined by the distance between opposite sides of at least one side wall. Optionally, the top end comprises a removable cap that seals the at least one vessel from the top end while in operation. Optionally, the cap may be a screw cap, a vacuum cup, a push cap, and so forth. The top end, the bottom end and the side wall are tightly sealed together to prevent the contents of the at least one vessel from leaving the vessel, unless the cap is removed (such as during harvesting). Optionally, the cap comprises air holes or a simple one-way air valve, to allow the air passing through the cyanobacterial biomass to escape via the top end of the vessel so as to not build up a pressure therein. Beneficially, the air escaping from the top end of the vessel comprises a significantly good concentration of oxygen that may be used directly or via an oxygen concentrator by humans for breathing. Optionally, the top end, the bottom end and the at least one side wall of the at least one vessel encloses a volume ranging between 100 ml and 3000 ml. Optionally, the volume of the at least one vessel is 1500 ml.

Optionally, the at least one vessel may have a shape, for example cylindrical, conical, cuboidal or cubical. In an embodiment, the at least one vessel is cylindrical in shape, with the top end, the bottom end, and a side wall connecting the top end and the bottom end. The at least one vessel may be fabricated of a material that is inert to the contents thereof. In an example, the material used for fabrication may be glass material, fibres, ceramic, plastic materials, stainless steel, other suitable metals or alloys, and/or combinations thereof. Moreover, the fabrication material is typically waterproof and strong enough to withstand abrasive effects of various biological, biochemical and/or mechanical processes, such as micro-organism concentrations, biomass productions, aeration forces, operating pressures, temperatures, and so forth. In an embodiment, the at least one vessel is fabricated from a transparent material, such as glass, fibre, plastic material or a combination thereof, to enable the user to view the growth of the cyanobacterial biomass therein. In another embodiment, the transparent fabrication material of the at least one vessel enables the cyanobacterial biomass to receive light from its surroundings (such as sunlight, lit room) for a continued growth thereof.

Moreover, the bottom end is a closed end consisting of the one-way air valve. Notably, the one-way air valve (also called ‘check-valve’) allows fluids, such as air, to flow in one direction but vitally closes to halt any backward motion thereof. In this regard, the one-way air valve opens when a higher pressure is provided on an input side of the one-way air valve rather than on an output side thereof. Moreover, the one-way air valve closes when the pressure is higher on the outlet side. Optionally, the one-way air valve is a metal, a rubber, a plastic material, or a combination thereof. Optionally, the one-way air valve is shaped as an annulus, a pin, and so on. The one-way air valve is complementary (in shape, in mode of operation, for example) with a corresponding air pump, such as the cultivation air pump and a harvesting air pump (as discussed later). It may be appreciated that a variety of one-way air valves exist and are known to a person skilled in the art, and thus has not been described in detail herein for the brevity of the present disclosure.

Moreover, the system comprises at least one light source that, when in operation, supplies light and heat to the at least one vessel, wherein the at least one light source is arranged on a side of the at least one vessel. The term “light source” as used herein refers to a source that emits light to provide illumination to the at least one vessel. It will be appreciated that light is a primary energy source for photosynthetic microorganisms. Naturally, the metabolic activity of microorganisms is largely driven by solar energy, therefore, in a closed environment like PBRs, such as consumer PBRs, the natural light is replaced by artificial light sources. Since cyanobacteria are photoautotrophic microorganisms, they require light to initiate photosynthesis. It will be appreciated that the cyanobacteria require a specific wavelength of light suitable for its growth. Optionally, the light source emits a visible light with its wavelength ranging from 380-750 nm. Optionally, the artificial light sources may be tungsten lamps, incandescent lights, fluorescent lights, light-emitting diodes (LED), and the like. Beneficially, the purpose of the light source is to provide illumination to the cells to initiate photosynthesis. Moreover, the at least one light source emits heat as a by-product. It will be appreciated that the at least one light source provides an optimum temperature necessary for a particular stage of growth of the cyanobacterial biomass

In this regard, the at least one light source is arranged on at least one side of the at least one vessel. Specifically, the arrangement of the at least one light source is based on the shape of the at least one vessel. In other words, the different shapes of the at least one vessel will have different arrangements of the at least one light source thereon. For example, on a cylindrical-shaped (or a conical-shaped) vessel, the at least one light source is arranged on a part (that may be considered a side) of the side wall. In another example, on a cuboidal shaped vessel, the at least one light source is arranged on at least one of the 4 faces of the side wall. In yet another example, on a frustum-shaped vessel, the at least one light source is arranged on at least one of the 4 faces of the side wall.

It will be further recognized that the light source may also provide part of a light absorption apparatus, in conjunction with a photodiode, for determining the concentration of cyanobacterial biomass in the vessel. Therefore, the light source may be supplied with electricity to alternate between this analytical function of determining concentration and the function of supplying an optimal intensity of light for growth.

Furthermore, the system comprises at least one photodiode that, when in operation, measures a cyanobacterial biomass concentration in the at least one vessel, wherein the at least one photodiode is arranged opposite to the at least one light source. The term “photodiode” as used herein refers to a semiconductor device that converts an incident light from the at least one light source into an electric energy (in the form of current or voltage). Moreover, the converted electric energy may be measured by an analogue to digital converter (ADC). The ADC may be communicably coupled to the microcontroller, or the ADC may be communicably coupled to the photodiode and be arranged to convert the measurement into a digital signal which is interpretable by the microcontroller, to determine the concentration of the cyanobacterial biomass (referred to as “cyanobacterial biomass concentration” hereafter). Alternatively, the converted electric energy measured with a multimeter or an LED indicator may be compared with a pre-determined value, then the energy provided to the light source opposite to the photodiode is correlated with pre-calibrated cyanobacterial biomass concentration levels corresponding to the energy values. The cyanobacterial biomass concentration relates to an optical density of the cyanobacterial biomass. Operatively, the optical density is deduced by the measured value obtained from the multimeter or an LED indicator, and translated into a gravimetric density as the weight of the cyanobacterial biomass in a given volume of the at least one vessel. Beneficially, the at least one photodiode is used to detect the amount of light passing through the culture at any given time to determine the concentration of the cyanobacterial biomass.

Typically, the at least one light source and the at least one photodiode is arranged on opposite sides of the at least one vessel. In this regard, the light intensity from the illuminated at least one light source is set to a maximum intensity and is made to fall incident on the at least one photodiode on the opposite side of the at least one light source. The incident light intensity is detected by the at least one photodiode, and a pre-defined value corresponding to the cyanobacterial biomass concentration is output by the at least one photodiode. Moreover, the incident light intensity is ensured to be the same between subsequent readings. This is achieved by measuring the current flowing through the at least one light source and maintaining the same current between subsequent readings. It will be appreciated that any decrease in the subsequent pre-defined values represents an increase in the optical density and in turn the gravimetric density of the cyanobacterial biomass.

It will be appreciated that the optical density of the cyanobacterial biomass has different light intensities at different cyanobacterial biomass concentrations for which the at least one photodiode has a pre-defined value. Furthermore, the optical density of the cyanobacterial biomass changes from a translucent shade to an opaque shade with the increasing gravimetric density of the cyanobacterial biomass. Moreover, the translucent shade denotes a stage when the cyanobacterial biomass is at an early stage of cultivation. During the translucent shade, the cyanobacterial biomass allows more light intensity to pass therethrough to be incident on the at least one photodiode. During the cultivation, the gravimetric density of the cyanobacterial biomass increases, thereby changing the translucent shade to the opaque shade. During the opaque shade, very little light intensity is able to pass through the cyanobacterial biomass to be incident on the at least one photodiode.

Optionally, the at least one photodiode is operatively coupled to an LED indicator. The at least one photodiode is operatively coupled to an LED indicator that indicates various growth stages of the cyanobacterial biomass. In this regard, the LED indicator provides a continuous ‘orange’ light to indicate that the biomass is growing, a flashing ‘orange’ light to indicate a stage where the cyanobacterial biomass is growing and requires user's attention, and a ‘green’ light to indicate that the cyanobacterial biomass is ready to harvest.

Furthermore, the system comprises a base unit configured to receive the bottom end of the at least one vessel. The term “base unit” as used herein refers to a docking station for the at least one vessel. In other words, the system is in operation when the one or more components are in a docking position, i.e., the at least one vessel is arranged on the base unit. In this regard, the base unit couples the at least one vessel, one or more means (such as, a cultivation air pump) for regulating growth conditions, and so forth, to a power supply. Optionally, the power supply is received from an external electric power source by a wireless or wired connection, for example a wireless resonant-inductive connection. Optionally, the power supply is received from a power source such as a battery. Specifically, the base unit supplies power to the PCB encircling the at least one vessel via at least one pogo electrical pin corresponding to the at least one vessel. Optionally, the base unit is designed as per the number of vessels per system. For example, the base unit could dock 2 vessels per system. Alternatively, the base unit could dock 1, 2, 3, 4 vessels, and so forth.

Furthermore, the system may comprise at least one heating element that, when in operation, supplies heat to the at least one vessel. The term “heating element” as used herein refers to a heating pad that converts electrical energy into heat. The at least one heating element may be positioned close to the at least one vessel such that the distance between the at least one heating element and the at least one vessel is minimum to obtain a maximum heat transmission into the cyanobacterial biomass. Typically, the at least one heating element provides an optimum temperature necessary for a particular stage of growth of the cyanobacterial biomass.

Furthermore, the system comprises a cultivation air pump that, when in operation, supplies air to the at least one vessel via the one-way air valve therein. The term “cultivation air pump” as used herein refers to an air pump for supplying air to the at least one vessel for aerating the cyanobacterial biomass therein. Moreover, the cultivation air pump is powered from the power supplied to the base unit, i.e., is mains powered. The cultivation air pump is connected via a length of silicone tubing to the tapered connection in the base unit. Moreover, the cultivation air pump comprises a cultivation air stone. The cultivation airstone incorporates a one-way air valve in its tapered lower extension. Thus, when the tapered (luer-type) connections of the vessel and base unit are separated for harvesting, the liquid contents of the vessel do not spill out. In this regard, the cultivation air pump connects to the at least one vessel via the one-way air valve at the bottom end of the at least one vessel. Specifically, the one-way air valve corresponding to the at least one vessel allows incoming air from the cultivation air pump to enter the at least one vessel, while halting the backward motion thereof. Notably, the supplied air is used to aerate the cyanobacterial biomass in the at least one vessel and to maintain an optimum pressure therein. Moreover, the air pump may supply, in the at least one vessel, an optimum amount of carbon dioxide and/or other gases necessary for the growth of the cyanobacterial biomass. Furthermore, the air supplied by the air pump enables the cells (cyanobacterial biomass) to circulate within the volume of the at least one vessel and get evenly exposed to the light therein. The cultivation airstone allows the air pumped into the vessel to form small bubbles such that an increased surface area of air contacts the cyanobacterial biomass. Optionally, the cultivation air pump is a piezoelectric air pump. The piezoelectric air pump is a compact, diaphragm-based pump specifically designed for built-in applications.

Optionally, the system further comprises an electrical circuitry for supplying power to the at least one light source, the least one photodiode, and the cultivation air pump. The electrical circuitry is a closed path in which electrons move to provide electric current to the base unit. As mentioned above, the one or more components receive power supply for respective operations thereof by the base unit. Beneficially, the electrical circuitry provides the constant voltage owing to the presence of a resistor, a capacitor, an inductor, and the like, connected in series or parallel connections.

Optionally, the system further comprises the microcontrollers configured to indicate, to a user, that the cyanobacterial biomass is ready to harvest when the cyanobacterial biomass concentration reaches a pre-defined value. The term “microcontroller” as used herein refers to a computational element that is operable to respond to and process instructions based on the growth stage of the cyanobacterial biomass and relay the information to the user. Optionally, the microcontrollers include but is not limited to, a controller, a processor, a microprocessor, a complex instruction set computing (CISC) microprocessor, a reduced instruction set (RISC) microprocessor, a very long instruction word (VLIW) microprocessor, Field Programmable Gate Array (FPGA) or any other type of controlling circuit, for example as aforementioned. Additionally, the one or more microcontrollers is arranged in various architectures for responding to and processing the instructions for determining the growth stage of the cyanobacterial biomass. Optionally, the microcontroller is a compact integrated circuit (IC) designed to govern a specific operation therein. Typically, the microcontroller includes a processor, a memory and input/output (I/O) peripherals on a single chip.

The microcontrollers may be communicably coupled to the wireless network including, but not limited to, Bluetooth®, Wireless Fidelity (Wi-Fi), Local Area Networks (LANs), Wide Area Networks (WANs), Metropolitan Area Networks (MANs), Wireless LANs (WLANs), Wireless WANs (WWANs), Wireless MANs (WMANs), the Internet, second generation (2G) telecommunication networks, third generation (3G) telecommunication networks, fourth generation (4G) telecommunication networks, fifth generation (5G) telecommunication networks and Worldwide Interoperability for Microwave Access (WiMAX) networks.

Optionally, the microcontrollers may follow a master-slave architecture, wherein the microcontrollers are arranged in the system as one or more master microcontroller and one or more slave microcontroller. In an embodiment, the master-slave architecture is implemented with 3 microcontrollers, i.e., one master microcontroller and two slave microcontrollers. The master microcontroller is more powerful and is configured to communicably couple to the wireless networks, to provide communication between the system and the user. The slave microcontrollers have a more basic feature set and each one controls the activity of the at least one vessel. Optionally, the slave microcontrollers are arranged in the housing that encircles the at least one vessel. The slave microcontrollers communicate with the master microcontroller via the wireless network. Optionally, an I2C communication protocol is used for communication between the master microcontroller and the slave microcontrollers, as it only requires 2 wires, thereby minimising the size and number of pins required from the contact connections.

Based on the master microcontroller's command, the slave microcontrollers measure density, recheck brightness settings, read temperatures and receive the results, and subsequently upload the above information to a database. In this regard, the slave microcontrollers communicate with the at least one light source, at least one photodiode, the cultivation air pump, and other sensors. Moreover, the slave microcontrollers perform the micro-level tasks, thereby leaving the master microcontroller to merely communicate with the application programming interface over wireless network, thus offering “smart features”. In this regard, the master microcontroller collects information from the slave microcontrollers and relays the collected information to a computing device (such as a user's mobile phone) that provides the application programming interface thereon. The information may be relayed as a notification that the cyanobacterial biomass (namely, spirulina) is ready to harvest. Optionally, the database could be on any server, cloud, drive, and the like from where the user can access the information through the application programming interface when required.

Optionally, the at least one light source and the at least one photodiode is arranged on at least one printed circuit board, wherein the printed circuit board and microcontroller are accommodated in a housing configured to partly cover the at least one vessel. The term “printed circuit board” as used herein refers to a thin board that provides a mechanical support and electrically connects the electrical or electronic components, such as the at least one light source and the at least one photodiode, using conductive elements, such as wires, pads, tracks, pathways and so forth, etched from copper sheets and laminated onto a non-conductive substrate. Moreover, the at least one PCB is connected to the microcontrollers to receive a command therefrom, based on the commands perform the desired activity. In this regard, for example, the at least one PCB instructs the at least one light source to regulate the light intensity. Subsequently, the at least one PCB instructs the at least one photodiode, upon receiving the incident light thereon, to determine the optical density of the cyanobacterial biomass.

Optionally, the at least one PCB has a form factor of a curved shape, flat rectangular shape, or any polygonal shape, arranged to be held by the housing. In an example, the curved form factor of the at least one PCB is implemented using flexible plastic substrates, such as fiberglass, polyimides, Polyether Ether Ketones (PEEK), a composite epoxy, or transparent conductive polyester films. Beneficially, such flexible plastic substrates allow bending of the measurement coil into the curved shape to be held by the housing.

The term “housing” as used herein refers to a protective layer that is configured to encircle (or surround) the at least one vessel at least partly or completely. The housing may be configured to at least partially surround an axially extending portion of the at least one vessel (for example, the housing may not be configured to cover the top end of the at least one vessel) and the bottom end of the at least one vessel. The housing contains at least one PCB comprising the at least one light source to illuminate the at least one vessel, the at least one photodiode to measure the cyanobacterial biomass in the at least one vessel, and other sensors for measuring various growth conditions in the at least one vessel. Moreover, the housing provides insulation and prevents moisture and other environmental factors to affect the at least one vessel, the contents of the at least one vessel, or the at least one PCB. While partially surrounding the at least one vessel, the housing may be configured to leave a part of the at least one side wall in order for the user to view the growth of the cyanobacterial biomass or the cyanobacterial biomass to receive light from surrounding (such as sunlight, lit room). Optionally, the housing has a tubular form-factor. Alternatively, the housing comprises any form-factor selected from any of: an elliptical, a cylindrical, a cuboidal, a conical or any other polygonal form-factor. It will be appreciated that the housing is designed based on a form factor of the at least one vessel to be held therein. Optionally, the housing may be fabricated from a plastic material (for example, polypropylene or polycarbonate), a silicone material, a rubber material, a ceramic material, a metal or alloy of metal (for example, aluminium), and so forth. In an instance, the housing is manufactured using a polymer. In another instance, the housing is manufactured using a metal alloy.

In an embodiment, the housing is attached to the base unit and is configured to receive the at least one vessel in the docking position. In another embodiment, the housing is removable (detachable) from the base unit as well as the at least one vessel. In this case, the harvesting of the cyanobacterial biomass is done conveniently.

Optionally, the cultivation air pump, the electrical circuitry, and the microcontrollers are accommodated in the base unit. Optionally, the arrangement of the aforesaid components in the base unit makes the system compact. Additionally, the power supply to the base unit is efficiently used to power up said components for their respective activities. Optionally, the base unit further comprises at least one pogo electrical pin for supplying electrical power to the at least one PCB when in docking position. It will be appreciated that the at least one pogo electrical pin corresponds to at least one vessel.

In an embodiment, the at least one PCB is arranged in the base unit and at least one PCB is arranged in the top end of the at least one vessel, such as the cap, opposite to the base unit. In such a case, the at least one light source is arranged on one of the at least one PCB and the at least one photodiode is arranged opposite to the at least one light source on the other at least one PCB. In such a case, the requirement for a housing can be eliminated.

Optionally, the system further comprises a harvesting arrangement for harvesting cyanobacterial biomass. The term “harvesting arrangement” as used herein refers to an arrangement configured for withdrawal of the grown cyanobacterial biomass. The disclosed harvesting arrangement is a compact, pressure-driven arrangement that harvests or withdraws fresh cyanobacterial biomass from the at least one vessel. In this regard, the at least one vessel is removed from the housing thereof and arranged on the harvesting arrangement for harvesting the cyanobacterial biomass. The harvesting arrangement comprises elements such as a double-ended harvesting vessel, a threaded mesh component, a mesh cap, and a top cap.

The harvesting arrangement may comprise a harvesting stand for receiving the at least one vessel from the top end thereof. The term “harvesting stand” as used herein refers to a rack for holding, supporting an external component, such as the at least one vessel, in a certain position. The harvesting stand may receive the at least one vessel in an inverted position of the at least one vessel. In this regard, the cap covering the top end of the at least one vessel may be replaced with the harvesting stand, such that the top end of the at least one vessel faces downward. It will be appreciated that in such orientation the content of the at least one vessel is inverted and occupies space near the top end leaving a space at the bottom end of the vessel. It will be appreciated that the content of the at least one vessel forms a water line near the bottom end of the at least one vessel. The harvesting stand may contain the harvesting mesh. The term “mesh component” as used herein relates to a perforated, sieve-like membrane that allows finer particles or liquids to pass therethrough while retaining coarser particles thereon.

Optionally, the harvesting mesh has an operational area in a range of 50×50 mm to 150×150 mm. The operational area of the harvesting mesh may typically be in a range from 50×50, 50×100, 70×100, 100×100 or 100×150 mm up to 50×100, 70×100, 100×100, 100×150 or 150×150 mm. In this regard, the length of the harvesting mesh may typically be in a range from 50, 60, 70, 80, 90, 100, 110, 120, 130 or 140 mm up to 60, 70, 80, 90, 100, 110, 120, 130, 140 or 150 mm, and width of the harvesting mesh may typically be in a range from 50, 60, 70, 80, 90, 100, 110, 120, 130 or 140 mm up to 60, 70, 80, 90, 100, 110, 120, 130, 140 or 150 mm. Optionally, the harvesting mesh is a square-shaped, a rectangle-shaped, or any polygonal shaped mesh. In an embodiment, the harvesting mesh could be square-shaped mesh having an operational area in a range of 100×100 mm. Optionally, the harvesting mesh could be circular-shaped mesh. In this regard the diameter of the circular-shaped mesh may be in a range of 50×150 mm. The diameter of the circular-shaped harvesting mesh may typically be in a range from 50, 60, 70, 80, 90, 100, 110, 120, 130 or 140 mm up to 60, 70, 80, 90, 100, 110, 120, 130, 140 or 150 mm. Moreover, the harvesting mesh is typically arranged near the top end of the at least one vessel for collecting the cyanobacterial biomass.

The harvesting arrangement may further comprise the harvesting air pump for supplying air into the at least one vessel via the one-way air valve, the harvesting air pump being configured to pressure the cyanobacterial biomass against a harvesting mesh arranged in the harvesting stand vessel for collecting the cyanobacterial biomass. The term “harvesting air pump” as used herein refers to a high-pressure air pump for supplying air to the at least one vessel for separating the dissolved nutrient growth media solution from the cyanobacterial biomass by applying a high pressure above a waterline in the inverted at least one vessel. In this regard, the harvesting air pump may connect to the at least one vessel via the one-way air valve at the bottom end of the at least one vessel. Specifically, the one-way air valve corresponding to the at least one vessel allows incoming air from the harvesting air pump to enter the at least one vessel, while halting the backward motion thereof. Optionally, the harvesting air pump may run continuously or intermittently, to collect the cyanobacterial biomass. The harvesting air pump may be a battery-powered air pump, different from the mains-powered cultivation air pump. Optionally, the harvesting air pump may be a diaphragm air pump.

It will be appreciated that the user (or the consumer) is able to cultivate and harvest 0.5-1 g of spirulina biomass in 1 day. Moreover, the user could harvest the fresh spirulina biomass using merely gravity, in less than a few minutes (such as 2 minutes) once a harvesting density is reached in the at least one vessel.

Optionally, the system further comprises a software module executable on the microcontrollers, which is trained using machine learning algorithms, to regulate growth conditions based on the cyanobacterial biomass concentration in the at least one vessel. Typically, the machine learning algorithms employ an artificial intelligence system. Throughout the present disclosure, the term “artificial intelligence system” as used herein relates to computationally intelligent system that combines knowledge, techniques, and methodologies for controlling a bot or other programmable components (such as the at least one light source, the cultivation air pump, and so forth) within a computing environment. Furthermore, the artificial intelligence system is configured to apply knowledge that can adapt itself and learn to do better in changing environments. Additionally, employing any computationally intelligent technique, the artificial intelligence system is operable to adapt to unknown or changing environments for better performance. The artificial intelligence system includes fuzzy logic engines, decision-making engines, pre-set targeting accuracy levels, and/or programmatically intelligent software. Artificial intelligence system in the context of the present disclosure relates to software-based algorithm, namely, the software module that is executable upon computing hardware, namely, the microcontrollers, and is operable to adapt and adjust their operating parameters in an adaptive manner depending upon information that is presented to the software module when executed upon the microcontrollers. Optionally, the artificial intelligence system includes neural networks such as recurrent neural networks, recursive neural networks, feed-forward neural networks, convolutional neural networks, deep belief networks, and convolutional deep belief networks; self-organizing maps; deep Boltzmann machines; and stacked de-noising auto-encoders. Specifically, the machine learning algorithms enable collating the growth data of all users' systems globally, using it to update the system and optimise the cyanobacterial biomass over time, leading to a network effect.

The term “software module” as used herein refers to a software program comprising executable instructions to perform one or more distinctive data processing operations. In an example, the distinctive data processing operations include, but do not limit to, receiving user input, optimizing and operating system components, such as, the at least one light source, the cultivation air pump, and so forth. The software module may be stored as an instruction (algorithm) on a computer-readable medium, such as in the microcontrollers, in the system and executed thereby to operate the at least one light source, the cultivation air pump to regulate growth conditions based on the cyanobacterial biomass concentration in the at least one vessel. Optionally, the software module is executable on the microcontrollers. The software module communicates information and commands to the microcontrollers. The received information and commands are used to program the microcontrollers one or more of the system components to optimize the system elements as required. Optionally, the software module is a set of one or more software applications. However, each software application serves a unique and separate operation, as mentioned above. Specifically, the software module performs supervisory monitoring, control, data analysis and optimisation of the one or more system components as required in a coordinated manner. Beneficially, such coordinated and optimal control actions ensure real-time monitoring and control of the system, thereby reducing frequent manual intervention by the user to ascertain the level of growth in the at least one vessel. Optionally, the software module may be pre-installed on the system or can be downloaded from a client network, a remote data storage, or internet. The software modules may be a System, Applications and Products (SAP) module, an enterprise resource planning (ERP) software, and so on.

In an embodiment, the machine learning algorithms employ supervised learning techniques, to regulate growth conditions based on the cyanobacterial biomass concentration in the at least one vessel. The term “supervised learning algorithm” refers to a learning technique employed by the software module to train the programmable components using labelled training dataset, structured training information or training with a desired output. Specifically, the training dataset employed for training the programmable components using supervised learning algorithms is classified and/or labelled. Alternately, the supervised machine learning algorithms analyse the labelled training dataset provided for training and further interpret the training dataset so as to sort the training data using predefined labels. In this regard, the machine-learning algorithms analyse a plurality of data sources to learn the different growth conditions and the required customizations performed in the past to regulate growth conditions. Herein, the software module employs previously received data of the regulated growth condition as a learning dataset for comparison with the currently received reading of the regulated growth condition. Notably, the machine learning algorithms compare the received reading of the regulated growth condition with previously received and verified readings of the regulated growth condition to validate the optimal regulated growth condition suitable for growth of a specific organism. In one example, the machine learning algorithms are used to determine whether a given temperature, light condition, incubation period will be suitable for a given organism's growth or not, based on the prior utilization of such conditions for the given organism. Therefore, if on a given day, the order of scanning the regulated growth condition changes or the intervals between each scan substantially change, the machine learning algorithms may raise a flag or an alarm to the user (or operator of the system).

In another embodiment, the software module is trained using an unsupervised machine learning algorithm, to regulate growth conditions based on the cyanobacterial biomass concentration in the at least one vessel. The term “unsupervised learning algorithm” refers to a learning technique employed by the software module to train the programmable components using unlabelled training dataset, unstructured training information or training without a desired output. Specifically, the training dataset employed for training the programmable components using unsupervised learning algorithms is neither classified nor labelled. Alternately, the unsupervised machine learning algorithms analyse the unlabelled training dataset provided for training and further interpret the training dataset so as to sort the training data without using predefined labels.

Optionally, the regulated growth conditions include:
growing the cyanobacterial biomass continuously;
a temperature in a range of 28 to 40° C.;
a photoperiod of 24 hours;
an illumination intensity in a range of 20 to 2500 μmol of photons/m²/s; and
a colour temperature in a range of 2000 to 7000K.

In this regard, the said regulated growth conditions are essential for growing a desired quality and quantity of the cyanobacterial biomass. The term “growing” as used herein refers to subjecting the cyanobacterial biomass to regulated growth conditions, such as temperature, illumination, and so forth, in the at least one vessel for a pre-defined incubation time. It will be appreciated that the incubation time is different for different organisms such as microalgae, cyanobacteria, plants, and the like. Optionally, the cyanobacteria reproduce by asexual reproduction, either by means of binary or by multiple fission in unicellular and colonial forms or by fragmentation and spore formation in filamentous species to produce the cyanobacterial biomass. It will be appreciated that the cyanobacterial biomass is continually grown. Additionally, an initial growth period of 1 week is required before the cyanobacterial biomass is ready for harvesting. Notably, the cyanobacterial biomass is grown continuously, and harvested daily.

It will be appreciated that different organisms such as microalgae, cyanobacteria, plants, and the like, grow at different temperatures. The temperature for growing the cyanobacteria may typically range for example from 28, 31, 35, 37, 38 or 39° C. up to 29, 30, 31, 33, 35 or 40° C. Moreover, besides the heat supply from the at least one light source, the temperature inside the at least one vessel may increase due to the respiration of the cyanobacterial biomass. The overall increase in the temperature of the at least one vessel may be sensed by a temperature sensor associated with the at least one vessel. Optionally, the temperature sensor could be arranged in the housing. Optionally, the temperature sensor is operatively coupled to the microcontrollers, and based on the sensed temperature of the at least one vessel, the microcontrollers regulate the at least one light source to accordingly supply heat the cyanobacterial biomass in the at least one vessel.

The term “photoperiod” as used herein refers to an interval in a 24-hour period during which the cyanobacterial biomass is exposed to light. In this regard, the at least one vessel is illuminated, via the at least one light source, for a period of 24 hours, i.e., continuously illuminated through the day and the night per day, for the cultivation of the cyanobacterial biomass.

The term “illumination intensity” (namely, the light intensity) as used herein refers to a measure of the power (namely, light) emitted by the at least one light source in a particular direction per unit area (defined by a solid angle (or field of view)) per unit time. The illumination intensity may typically range from 20, 30, 40, 50, 100, 200, 300, 400, 500, 1000, 1500 or 2000 μmol of photons/m²/s up to 30, 40, 50, 100, 200, 300, 400, 500, 1000, 1500, 2000 or 2500 μmol of photons/m²/s. It will be appreciated that the wavelength of the light is in the Photosynthetically Active Radiation (PAR) range that reaches one square meter per second. The PAR range is the spectral range (wave band) of solar radiation from 400 to 700 nanometres that photosynthetic organisms may use in the process of photosynthesis. Moreover, the illumination intensity varies with a change in the cyanobacterial biomass concentration. In other words, the illumination intensity has a specific value for a given stage of growth of the cyanobacterial biomass.

The term “colour temperature” as used herein refers to a measure of the warmth or coolness (namely, colour) of a light source. It will be appreciated that different light sources, such as tungsten lamps, incandescent lights, fluorescent lights, light-emitting diodes (LED) and the like, have different colour temperatures. The colour temperature for the cultivation of the cyanobacterial biomass typically ranges from 2000, 3000, 4000, 5000 or 6000K up to 3000, 4000, 5000, 6000 or 7000K.

Optionally, one or more components of the system are fabricated using at least one of: an injection-moulding technique, a printing technique, an etching technique. The injection-moulding technique refers to a manufacturing process of injecting molten material into a mould for producing parts of the system. The injection moulding could be performed using a variety of materials suitable for fabrication of different components of the system. Optionally, fabrication material may be selected from metals or metal alloys, glass, elastomers, thermoplastic, thermosetting polymers, and the like. The printing technique is typically a multidimensional printing process based on additive manufacturing for creating a three-dimensional object layer-by-layer using a computer created design. The etching technique is used in microfabrication to chemically remove layers from the surface of a wafer (such as a slice of a semiconductor) during manufacturing of PCBs for example. Beneficially, by employing said fabrication techniques, the system is manufactured at low costs, thereby appealing to a wide range of consumers.

The present disclosure also relates to the application programming interface as described above. Various embodiments and variants disclosed above apply mutatis mutandis to the application programming interface.

Herein, the term “application programming interface” refers to an application program or a computer program designed for performing a specific task. The data is translated in such a manner that it is decoded by the computing device and/or the microcontrollers. Moreover, the application programming interface may be open source, feature-rich, and includes various tools, such as animation, simulation, rendering, motion tracking, and so on. Additionally, the application programming interface may be developed and used to support ballistic, electromagnetic and geometric analysis tools, concentration sensors, temperature sensors, and the like.

Throughout the present disclosure, the term “computing device” as used herein refers to electronic device associated with (or used by) a user, which is capable of enabling the user to perform specific tasks associated with the aforementioned method and system. Furthermore, the computing device is intended to be broadly interpreted to include any electronic device that may be used for voice and/or data communication over a wired or wireless communication network. Examples of a computing device include, but are not limited to, cellular phones, personal digital assistants (PDAs), handheld devices, wireless modems, laptop computers, personal computers, etc. Additionally, the computing device includes a display, a camera, a memory, a processor, a communication interface. Moreover, the computing device is configured to host the application programming interface thereon to support and/or enable the operation of the system. Specifically, the application programming interface logs in to display the computing device to access the communication interface and display a virtual system. The application programming interface enables sending commands to the microcontrollers to control, configure or orchestrate one or more programmable components, such as the at least one light source, the cultivation air pump, and so forth. Herein, the term “user” as used herein refers to any entity such as a person (i.e., human being) or a virtual program (such as, an autonomous program or a bot) that is associated with or operates the user device.

Optionally, the communication interface includes, but is not limited to, microphone, display screen, touch screen, optical markers, and speakers. The display screen is typically large enough to show in large size (namely, clearly) the text and graphics, comprising pictures and/or videos. Examples of the display screen include, but are not limited to, a Liquid Crystal Display (LCD), a Light-Emitting Diode (LED)-based display, an Organic LED (OLED)-based display, a micro OLED-based display, an Active Matrix OLED (AMOLED)-based display, and a Liquid Crystal on Silicon (LCoS)-based display. The microphones may be used to receive (or record) audio streams. Further, the audio streams from the subject may be sent to processing arrangement in real time. Optionally, the audio streams may be pre-recorded by the subject using the microphone for play-back using the speaker, as required. Moreover, the speaker may be used to provide information about the growth of cyanobacterial biomass in the at least one vessel, or providing instructions to the subject.

Moreover, the application programming interface is communicably coupled with the microcontrollers. Notably, the data communication is established between two or more components over a network interface. The network interface may typically be an individual network, or a collection of individual networks, interconnected with each other and functioning as a single large network. Such individual networks may be wired, wireless, or a combination thereof. Examples of such individual networks include, but are not limited to, Local Area Networks (LANs), Wide Area Networks (WANs), Metropolitan Area Networks (MANs), Wireless LANs (WLANs), Wireless WANs (WWANs), Wireless MANs (WMANs), the Internet, second generation (2G) telecommunication networks, third generation (3G) telecommunication networks, fourth generation (4G) telecommunication networks, and Worldwide Interoperability for Microwave Access (WiMAX) networks. Optionally, the network interface is employed by the application programming interface integrated with the computing device to contact the communication interface.

The application programming interface comprises smart features. The smart features include reporting (namely, signal) of growth state and culture health to the user, the ability to control some growth parameters including, but not limited to, the regulated growth conditions, and general control of the system through the application programming interface. Specifically, such reporting and controls are facilitated by the microcontrollers (master and slave architecture discussed above) which are internet connected. The term “signal” as used herein refers to a function that conveys (namely, carries) information about a phenomenon.

Typically, the signal could be a voltage, a current, or an electronic wave that varies as a function of time. Moreover, the signal can be a light, an audio, a video, a speech, an image, a vibration, a motion, a temperature, a pressure, a position, a sonar, a radar-related, and so forth. Specifically, the signal is the observable change in a quality or quantity that is conveyed to a user of the system. The application programming interface enables the computing device to alert users to any issues that occur during the growing cycle, via on-board sensing in the at least one printed circuit board and enables users to receive notifications when the cyanobacterial biomass is ready to harvest. More specifically, signals are typically provided by sensors specific for observing said change in the quality or quantity, subsequently, signals are converted to another suitable form of energy using a transducer. Optionally, an LED indicator provided on the system, such as on the at least one vessel, also communicates the current status of the growth of the cyanobacterial biomass to the user. In this regard, the signal is a light signal that changes from one colour (such as ‘orange’) to another (such as ‘green’) based on the density of the cyanobacterial biomass in the at least one vessel, wherein the ‘orange’ colour light signals a low density and the ‘green’ colour light signals a desired density of the cyanobacterial biomass that is ready for harvesting.

Optionally, the application programming interface allows real-time growth tracking, where users can view a visualised representation of the at least one vessel's productivity over a growing cycle. Optionally, the microcontrollers relay information related to the at least one vessel's productivity over a growing cycle to the computing device via their home Wi-Fi connection, for example, where they will be notified that the cyanobacterial biomass (spirulina) is ready to harvest.

Optionally, the application programming interface is configured to switch the system from pre-defined regulated growth conditions to a ‘Holiday Mode’. Specifically, with the ‘Holiday Mode’ within the application programming interface the users will be able to set the status of their system in such a way that all light and heat supply will be reduced to levels which minimise the growth rate of the cyanobacterial biomass. More specifically, ‘Holiday Mode’ reduces the growth rate of the cyanobacterial biomass so that it is sustained for many weeks.

The present disclosure also relates to the method as described above. Various embodiments and variants disclosed above apply mutatis mutandis to the method.

The term “receiving” as used herein refers to a docking of the at least one vessel on the base unit such that the bottom end of the at least one vessel is arranged on a specific spot on the base unit. While receiving it is important that the at least one vessel is arranged such that the one-way air valve at the bottom end of the at least one vessel is aligned with the cultivation air pump, and the at least one vessel housing is aligned with a corresponding pogo electrical pin. The term “powering up” as used herein refers to providing an electrical supply to the one or more electrical or programmable components of the system. It will be appreciated that powering up said components could be achieved using an external power supply or an internal power source (such as a battery, coils) within the base unit. The term “predefined value” as used herein refers to the amount of the cyanobacterial biomass harvested from the system of different volumes. For example, a system with a volume or capacity of 1.5 litres could cultivate and harvest 1 g of the cyanobacterial biomass.

Optionally, the method further comprises harvesting the cyanobacterial biomass, wherein harvesting comprises
pouring a set volume of spirulina culture from the cultivation vessel into the harvesting vessel with mesh component attached to separate cyanobacterial biomass from growth medium;
placing a mesh cap over a mesh component to seal the bottom of the harvesting vessel; and
collecting the cyanobacterial biomass by pouring fresh water into the harvesting vessel, attaching the top cap and shaking.

It will be appreciated that in furtherance to the above, the harvesting vessel is inverted. Thereafter, the mesh component and the mesh cap are removed, and the cyanobacterial biomass is ready to be consumed. Notably, the cyanobacterial biomass is provided for consumption in the form of a liquid. Optionally, the method further comprises training a software module, executable on the microcontrollers, using machine learning algorithms, to regulate growth conditions based on the cyanobacterial biomass concentration in the at least one vessel.

Optionally, the regulated growth conditions include:
growing the cyanobacterial biomass continuously;
a temperature in a range of 28° C. to 40° C.;
a photoperiod of 24 hours;
an illumination intensity in a range of 20 to 2500 μmol of photons/m²/s; and
a colour temperature in a range of 2000 to 7000K.

Optionally, measuring the cyanobacterial biomass within a range of 0-1.5 g/L comprises:
a) arranging the at least one light source and a first photodiode on opposite sides of the at least one vessel;
b) illuminating the at least one vessel with the at least one light source, on one side of the at least one vessel, with a maximum light intensity;
c) measuring, using the first photodiode on the opposite side of the illuminated at least one light source, a unique value corresponding to the cyanobacterial biomass concentration at a given time;
d) determining, based on the unique value, a density of the cyanobacterial biomass at the given time; and
e) indicating to a user, via the microcontrollers, the density of the cyanobacterial biomass at the given time.

Optionally, measuring the cyanobacterial biomass within a range of 1-3.5 g/L comprises:
a) arranging a secondary light source and opposite to a second photodiode on a printed circuit board;
b) illuminating within the at least one vessel with the secondary light source, with a maximum light intensity;
c) measuring, using the second photodiode on the printed circuit board, a unique value corresponding to the cyanobacterial biomass concentration at a given time;
d) determining, based on the unique value, a density of the cyanobacterial biomass at the given time;
e) indicating to a user, via the microcontrollers, the density of the cyanobacterial biomass is equal to a harvesting density of the cyanobacterial biomass, wherein the harvesting density corresponds to a set value measured by the at least one photodiode.

In this regard, the at least one light source is illuminated with a maximum light intensity that is measured using the at least one photodiode opposite to the at least one light source. It will be appreciated that the at least one light source on one side of the at least one vessel is illuminated and the at least one light source on the side opposite to the illuminated at least one light source (that also bears at least one photodiode) is kept ‘OFF’. In this regard, the at least one light source on either side of the at least one vessel are first turned off and then only one side of the at least one vessel is illuminated by turning ‘ON’ the at least one light source, while the at least one photodiode on the opposite side is configured to measure the light intensity thereof. The term “maximum light intensity” as used herein refers to an intensity of light that actuates the at least one photodiode when the light is incident thereupon. The maximum light intensity is fixed based on a maximal capacity of outputting light of the at least one light source. Herein, the maximal capacity of the at least one light source is observed based on electronic and safety restrictions, regardless of a given stage of growth of the cyanobacterial biomass. Upon actuation, the at least one photodiode provides the unique value corresponding to the cyanobacterial biomass concentration at a given time (stage of growth). It will be appreciated that the maximum light intensity is kept constant at every measurement cycle to measure a particular cyanobacterial biomass concentration during each measurement cycle. Notably, the maximum light intensity is also kept constant during the measurement cycle. The term “unique value” as used herein refers to a value generated by the at least one photodiode for a unique cyanobacterial biomass concentration, for example, the unique value generated for each value of the cyanobacterial biomass concentration in the at least one vessel. It will be appreciated that a unique value is generated for each of these densities, wherein the unique value decreases as the cyanobacterial biomass concentration in the at least one vessel increases.

It will be appreciated that the density is checked twice here. Initially, the system to measure cyanobacterial biomass within a range of 0-1.5 g/L is used, and thereafter, the system to measure cyanobacterial biomass within a range of 1-3.5 g/L is used. Optionally, each measurement cycle comprises such double density checking. Optionally, a length of each measurement cycle lies in a range of 50-600 seconds. Optionally, firstly, the system to measure cyanobacterial biomass within a range of 0-1.5 g/L is used if the density of the cyanobacterial biomass is within 1 g/L. If the measured density is above 1 g/L, then the system to measure cyanobacterial biomass within a range of 1-3.5 g/L is also used simultaneously. Notably, at lower densities, i.e., less than 1 g/L, there is one density measurement per measurement cycle, and at higher densities, i.e., more than 1 g/L, there are two density measurements per measurement cycle. Herein, 1 g/L is taken as a threshold value such that the two density measurements have an overlapping threshold range (i.e., 1 g/L-1.5 g/L). Since a user's interaction with the cyanobacterial biomass cannot be anticipated, such double density checking is performed since the density of the cyanobacterial biomass is unknown in each measurement cycle. Beneficially, this saves time and effort since identification of a lower density is easily decipherable.

Moreover, based on the unique value, the density, namely the optical density, of the cyanobacterial biomass at the given time is determined. The optical density is the measure of absorbance of light by the at least one photodiode as limited by the cyanobacterial biomass concentration (optical density being indirectly proportional to the cyanobacterial biomass concentration). The density is efficiently determined by keeping the amount of light emitted constant, with the maximum light intensity for example, and measuring the difference in the incident light on the at least one photodiode.

Furthermore, the determined density is conveyed by the at least one photodiode to the microcontrollers. Optionally, the user may be notified through the application programming interface, communicably coupled to the microcontrollers, regarding the density of the cyanobacterial biomass at every stage, i.e., at a real-time or after pre-defined intervals. It will be appreciated that besides the notification at real-time or pre-defined intervals, the at least one photodiode is configured to notify the user regarding the harvesting density of the cyanobacterial biomass.

The term “harvesting density” as used herein refers to a density at which the cyanobacterial biomass is ready to harvest. It will be appreciated that the harvesting density corresponds to a set value measured by the at least one photodiode. The term “set value” as used herein refers to a minimum value generated by the at least one photodiode for a maximum cyanobacterial biomass concentration, namely 0-3.5 g/L, in the at least one vessel. Notably, the set value is minimum since a very small fraction of light is incident on the at least one photodiode when the cyanobacterial biomass concentration is very high or at a maximum density. It will be appreciated that when the user is notified, via the microcontrollers, that a set value has reached it indicates that the cyanobacterial biomass is ready to harvest. Moreover, per period, the cyanobacterial biomass concentration is only measured once, i.e., during the maximum light intensity, for a given stage of growth of the cyanobacterial biomass.

Optionally, the method further comprises determining an optimum light intensity for the next stage of growth, the method comprising:
a) turning off the at least one light source;
b) illuminating the at least one vessel with the at least one light source with an incremental light intensity until a pre-defined photodiode value is measured using the at least one photodiode on the opposite side of the illuminated at least one light source;
c) determining a light intensity resulting in the pre-defined photodiode value as the optimum light intensity for next stage of growth;
d) allowing the cyanobacterial biomass to grow for a pre-defined period with the optimum light intensity; and
e) repeating steps (a) to (d) until the density of the cyanobacterial biomass is equal to a harvesting density of the cyanobacterial biomass as obtained from cycle of density detection, wherein the harvesting density corresponds to a set value measured by the at least one photodiode.

In this regard, it will be appreciated that for a particular stage of growth (namely, culture density), there is a unique emitted light intensity by the at least one light source illuminated on one side of the at least one vessel, which results in a pre-defined photodiode value generated by the at least one photodiode on the opposite side of the illuminated at least one light source. The pre-defined photodiode value corresponds to a specific intensity of light incident on the at least one photodiode, hence determining the appropriate light intensity for the next growing period. The term “optimum light intensity” as used herein refers to an intensity of light that is required to be emitted across the culture for the next period, in order for the at least one photodiode to generate the pre-defined photodiode value, which is not equal to the set value, i.e., the value at which harvesting could be performed. The optimum light intensity is achieved by stepwise increasing the light intensity emitted by the at least one light source until the at least one photodiode generates a pre-defined photodiode value corresponding to a given cyanobacterial biomass concentration at a given time. The final light intensity that results in generation of the pre-defined photodiode value is determined as the optimum light intensity and is maintained constant for the next stage of growth, wherein the next stage of growth allows the cyanobacterial biomass to grow with the optimum light intensity for a pre-defined period of for example 120 seconds. Moreover, the cycle of density detection as discussed above and the cycle of illumination intensity setting as discussed herein is repeated until the density detection system outputs a photodiode reading which is equal to the set value, i.e., the value which indicates that the cyanobacterial biomass is ready to harvest. Moreover, at this point, the cycle of illumination intensity setting is stopped.

Optionally, the method comprises operating the microcontroller, based on a signal from an application programming interface communicably coupled with the microcontrollers, for regulating operation of the at least one light source, the at least one photodiode, the electrical circuitry, and the cultivation air pump.

The present disclosure also relates to the composition as described above. Various embodiments and variants disclosed above apply mutatis mutandis to the composition.

The composition comprises the cyanobacterial biomass at a particular growth stage, preferably at the log phase or stationary phase of growth. The cyanobacterial biomass composition typically consists of nutrients such as proteins, carbohydrates and lipids as measured during the stationary phase of growth. Moreover, an elemental analysis may also be performed during the lag and the log phases of growth to contribute to a deeper understanding of the cyanobacterial biomass composition. Beneficially, the cyanobacterial biomass is nutritious, comprising high protein and fibre content in addition to a range of vitamins and minerals, both in its fresh as well as dry state.

Optionally, the composition is used as a food or food ingredient. In this regard, the term “food” or “food ingredient” as used herein refers to a nutraceutical product and/or a dietary supplement. For example, the food is freshly cultivated and harvested spirulina. The fresh spirulina paste could be used to add to smoothies, juices, sauces, salad dressings, protein shakes, a glass of water, and so forth. Optionally, the food may be an additive feed supplement for poultry, water animals, and so forth. Optionally, the food may be a 1 g of the cyanobacterial biomass.

Optionally, the composition comprising the cyanobacterial biomass is employed as an ingredient in at least one of: human foods, human nutraceutical preparations or formulations, animal feeds, drug compositions, cosmetics, personal care compositions, personal care devices. The food for humans can include but is not limited to bakery products, pastas, cereals, cereal bars, confections, sauces, soups, dairy substitutes, frozen desserts, ice creams, yoghurts, smoothies, creams, spreads, salad dressings, mayonnaises, food garnishing and seasoning, candies, gums, jellies, vape liquid and so forth. Moreover, the cyanobacterial biomass can be used as an ingredient for example in nutraceutical preparations and formulation for humans, pharmaceutical compositions, cosmetics, personal care compositions, personal care devices and so forth. The nutraceutical preparations and formulation comprise, for example, nutritional supplements, hormone tablets, digestive capsules, tablets, powders, oils and the like. The cosmetic formulations may employ use of the cyanobacterial biomass or specific extracts derived therefrom, for example, in lipsticks, powders, creams, exfoliants, facial packs, and so forth. The personal care compositions and personal care devices comprise toothpastes, mouthwash, hand-wash, body-wash, body soaps, shampoos, oils, sun-creams, after-sun creams, sunblock and so forth. The pharmaceutical compositions include any type of compositions known to the skilled person for the delivery of medicaments, including bioactives, vaccines and delivery vehicles for other recombinant proteins and enzymes.

### DETAILED DESCRIPTION OF THE DRAWINGS

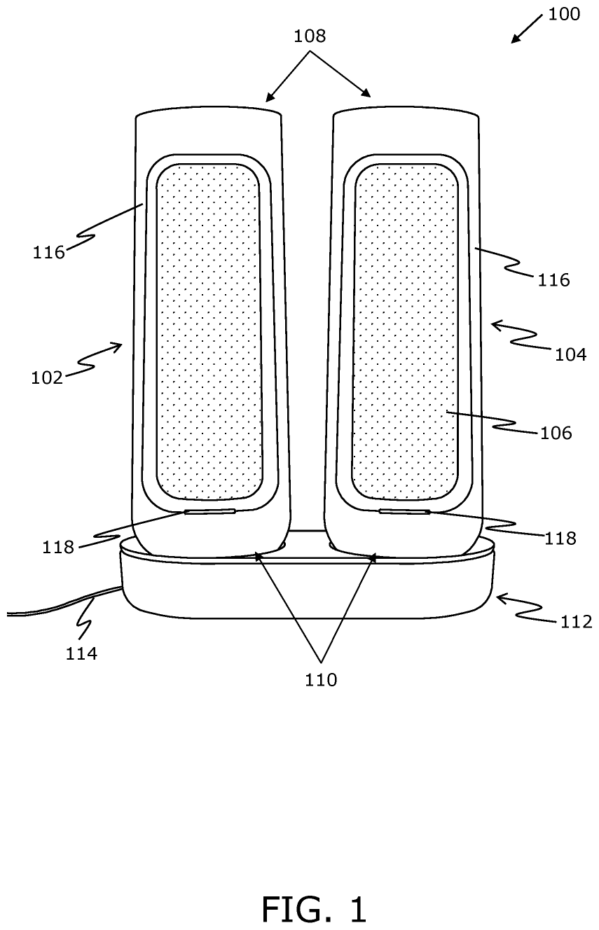

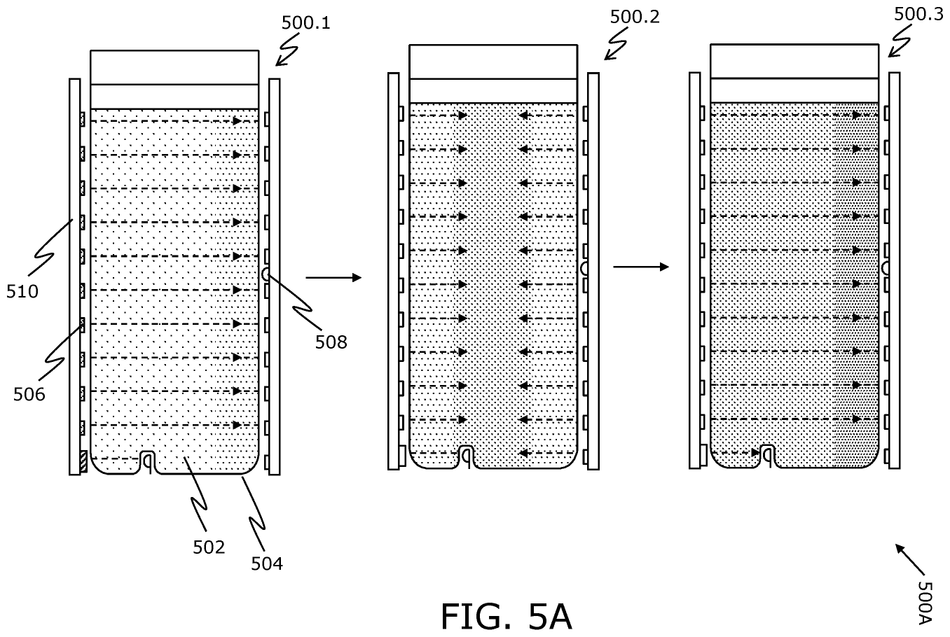

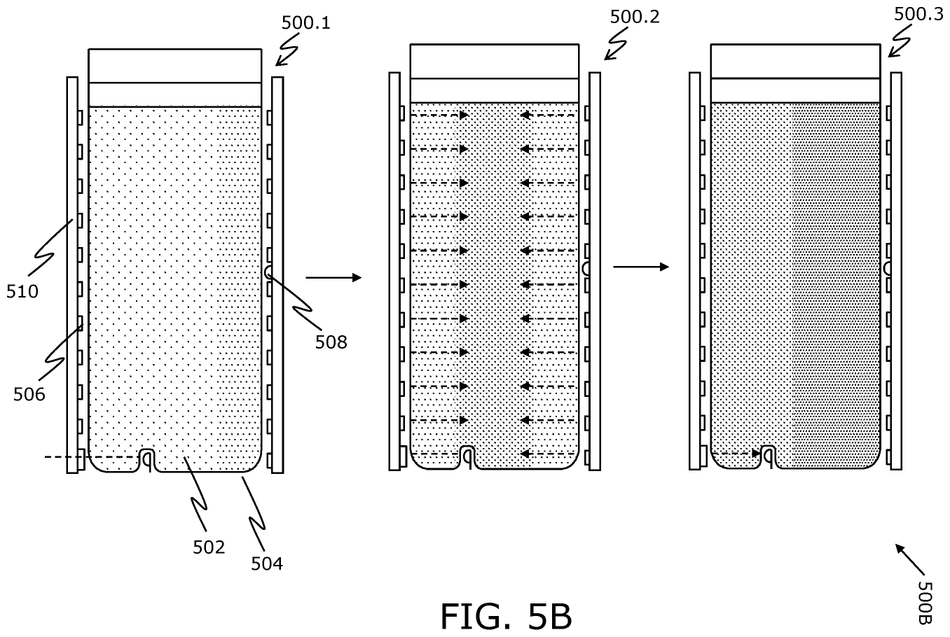

Referring to FIG. 1, illustrated is a system 100 for cultivating and harvesting a cyanobacterial biomass, in accordance with an embodiment of the present disclosure. The system 100 comprises at least one vessel, such as the vessels 102 and 104, that, when in operation, grows the cyanobacterial biomass 106 in a nutrient growth media under regulated growth conditions. The at least one vessel, such as the vessels 102 and 104, have a top end 108, a bottom end 110, at least one side wall 112 connecting the top end 108 to the bottom end 110, and a one-way air valve (not shown) at the bottom end 110. The system 100 comprises at least one light source (as shown in FIG. 5) that, when in operation, supplies light and heat to the at least one vessel, such as the vessels 102 and 104, wherein the at least one light source is arranged on a side (as shown in FIG. 5) of the at least one vessel, such as the vessels 102 and 104. The system comprises at least one photodiode (as shown in FIG. 5) that, when in operation, measures a cyanobacterial biomass concentration in the at least one vessel, such as the vessels 102 and 104, wherein the at least one photodiode is arranged opposite to the at least one light source.

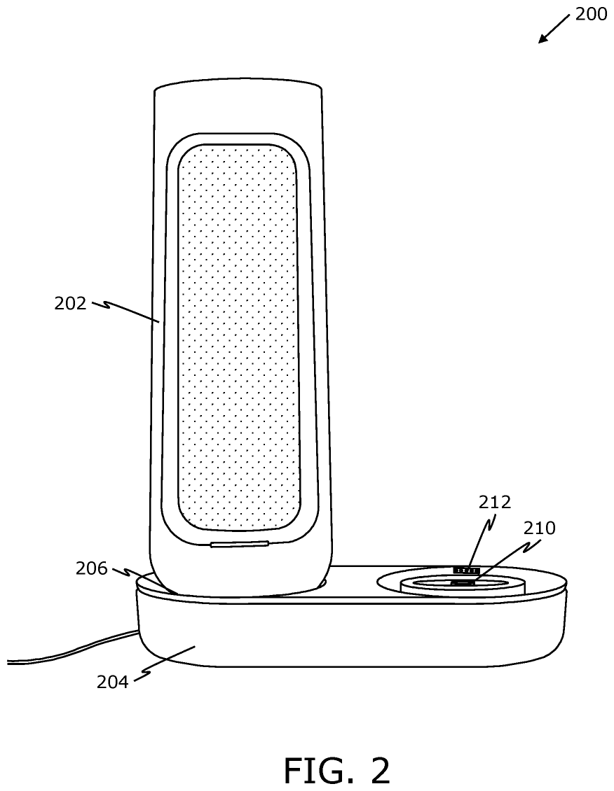

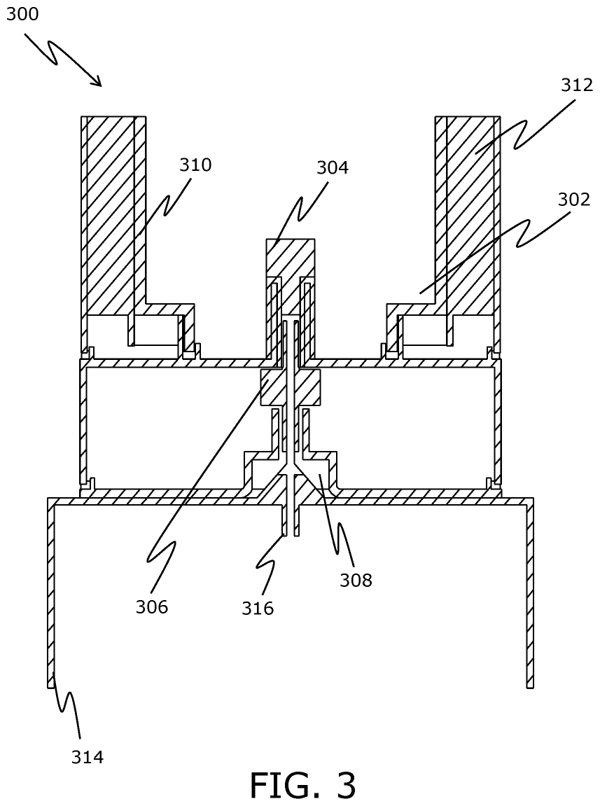

Moreover, the system 100 comprises a base unit 112 configured to receive the bottom end 110 of the at least one vessel, such as the vessels 102 and 104. The system comprises a cultivation air pump (as shown in FIG. 2 or 3) that, when in operation, supplies air to the at least one vessel, such as the vessels 102 and 104, via the one-way air valve therein.

As shown, the system 100 further comprises an electrical circuitry 114 for supplying power to the at least one light source, the at least one photodiode, and the cultivation air pump. The system 100 further comprises microcontrollers (not shown) configured to indicate, to a user, that the cyanobacterial biomass 106 is ready to harvest, when the cyanobacterial biomass concentration reaches a pre-defined value.

As shown, the at least one light source and the at least one photodiode is arranged on at least one printed circuit board (as shown in FIG. 5), wherein the printed circuit board and microcontroller are accommodated in a housing 116 configured to partly cover the at least one vessel, such as the vessels 102 and 104. Moreover, the cultivation air pump, the electrical circuitry, and the microcontrollers are accommodated in the base unit 112.

Moreover, the at least one vessel, such as the vessels 102 and 104, comprises coloured LED indicator 118 thereon, which is used to communicate one of three growing stages to the user: growing (orange light); growing but requires attention/tasks to be completed (orange flashing light); and ready to harvest (green).

Referring to FIG. 2, illustrated is a perspective view of the system 200 for cultivating and harvesting a cyanobacterial biomass, in accordance with an embodiment of the present disclosure. As shown, the system 200 comprises a vessel 202 and a base unit 204. The base unit 204 is configured to receive the bottom end 206 of the at least one vessel 202. Moreover, the base unit 204 comprises at least one depression therein to receive the bottom end 206 of the at least one vessel 202. The base unit 204 comprises at least one cultivation airstone 210, at least one cultivation air pump (not shown), microcontrollers (not shown), an electrical circuitry (not shown), and at least one pogo pin 212 for providing electricity to the at least one printed circuit board (not shown).

Referring to FIG. 3, illustrated is a cross-sectional view of the base unit 300 and a lower part of the vessel 302, in accordance with an embodiment of the present disclosure. Specifically, the figure illustrates a cross-sectional view of the base unit 300 and a lower part of the vessel 302 having an airflow connection with the cultivation airstone 304. The base unit 300 is configured to receive a bottom end of a vessel 302. As shown, the base unit 300 comprises a cultivation airstone 304, the electrical circuitry (not shown), and the microcontrollers (not shown). The cultivation airstone 304 incorporates a one-way air valve 306 and a silicone airflow connection component 308 in its tapered lower extension. Thus, when the tapered (luer-type) connections of the vessel 302 and base unit 300 are separated for harvesting, the liquid contents of the vessel 302 do not spill out. A cultivation air pump (not shown) is connected to the silicone airflow connection component 308 in the base unit 300. Moreover, the cultivation air pump is mains powered. The cultivation airstone 304 allows the air pumped into the vessel 302 to form small bubbles such that an increased surface area of air contacts the cyanobacterial biomass. The base unit 300 further comprises a glass cultivation vessel 310, from which a set volume of spirulina culture is poured into the vessel 302. The glass cultivation vessel 310 is encased in a cultivation vessel casing 312. The base unit 300 is encircled in a base housing 314. The air is received at the silicone airflow connection component 308 via a base airflow receiving component 316 which receives the air and forwards it into the base unit 300.

Additionally, the base unit 300 comprises pogo electrical pins (not shown) at the backside thereof, to power the at least one printed circuit board that encircles the vessel 302, allowing light to be delivered to the cyanobacterial biomass. It will be appreciated that each pogo electrical pin powers the at least one printed circuit board that encircles the vessel 302.

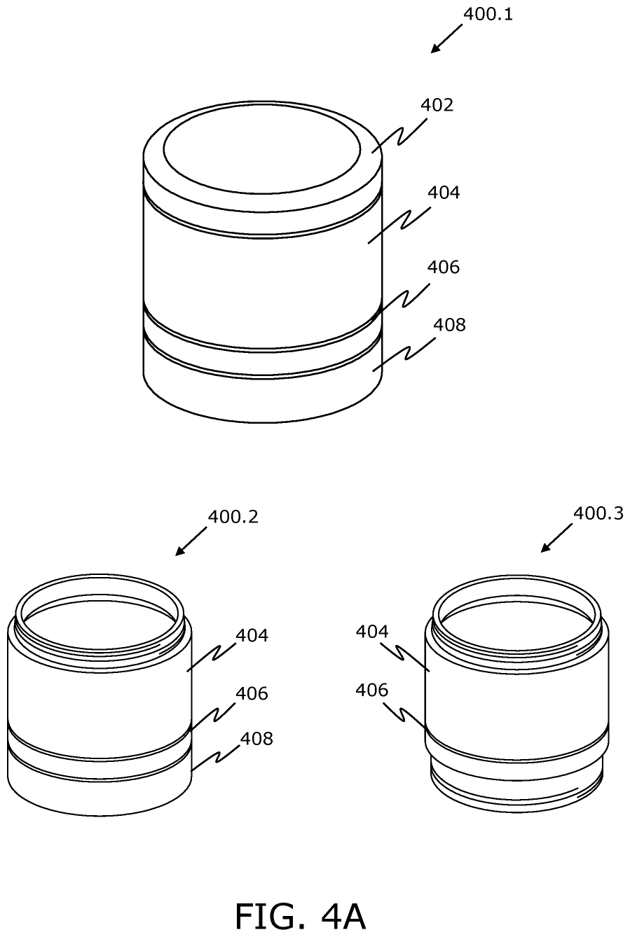

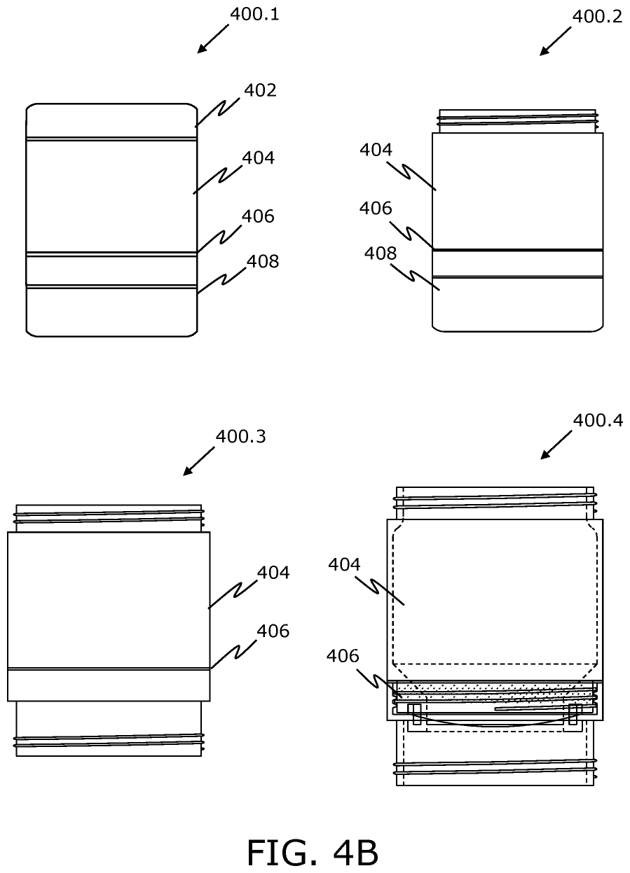

Referring to FIGS. 4A and 4B, illustrated are schematic illustrations of the harvesting arrangement 400.1, 400.2, 400.3 and 400.4 (hereinafter, collectively referred to as 400) for harvesting cyanobacterial biomass, in accordance with an embodiment of the present disclosure. The harvesting arrangement 400 comprises a top cap 402, a double-ended harvesting vessel 404, a threaded mesh component 406, and a mesh cap 408. The double-ended harvesting vessel 404 is utilized for receiving at least one set volume of spirulina culture. The threaded mesh component 406 is attached to the double-ended harvesting vessel 404 for separating the cyanobacterial biomass from the growth medium via filtration. The mesh cap 408 is utilized to stop the leaking of liquid while the double-ended harvesting vessel 404 is being shaken for the mixing of the harvested cyanobacterial biomass from the threaded mesh component 406 into a liquid drink. The top cap 402 is utilized for the double-ended harvesting vessel 404 to stop the leaking of liquid while the harvesting arrangement 400 is being shaken for the mixing of the harvested cyanobacterial biomass from the threaded mesh component 406 into a liquid drink. The harvesting arrangement 400 enables harvesting daily servings of fresh cyanobacterial biomass from the system, such as the system 100 of FIG. 1, which is compact and may fit on the countertop of the user's kitchen for example.

FIG. 4A illustrates a top perspective view of the harvesting arrangement 400 for harvesting cyanobacterial biomass, and FIG. 4B illustrates a side view of the harvesting arrangement 400 for harvesting cyanobacterial biomass. Moreover, as shown in FIGS. 4A and 4B, the harvesting arrangement 400.1 is shown with all components as explained above, the harvesting arrangement 400.2 is shown with the top cap 402 removed, and the harvesting arrangements 400.3 and 400.4 are shown with both the top cap 402 and the mesh cap 408 removed.

Referring to FIGS. 5A and 5B, illustrated are schematic illustrations of different stages of a density detection system 500A, 500B (hereinafter collectively referred to as 500), in accordance with an embodiment of the present disclosure. The density detection system 500 is operable to detect the cyanobacterial biomass 502 concentration in the at least one vessel 504. The at least one vessel 504 is encircled with at least one light source 506 and at least one photodiode 508, wherein the at least one photodiode 508 is arranged opposite to the at least one light source 506. The at least one light source 506 and the at least one photodiode 508 are arranged on at least one printed circuit board 510, wherein the at least one printed circuit board 510 is accommodated in the housing encircling the at least one vessel 504.

In this regard, with respect to the density detection system 500.1, starting with the at least one light source 506 ‘OFF’, the at least one light source 506 is turned ‘ON’. Subsequently, the light intensity is increased until the at least one photodiode 508 on the opposite side outputs a unique value corresponding to a unique cyanobacterial biomass 502 concentration in the at least one vessel 504 at a given stage of growth. The light intensity required to reach the unique value is recorded and correlated with a cyanobacterial biomass concentration. It is then used to grow the cyanobacterial biomass 502 for a pre-defined period, such as 2 minutes for example at that fixed light intensity, for a subsequent (namely, next) stage of growth. The operatively coupled at least one light source 506 and the at least one photodiode 508 allows for calculating the total cyanobacterial biomass 502 present in the at least one vessel 504 at a given stage of growth. Thereafter, with respect to the density detection system 500.2, the set value is used to set the brightness of the at least one light source 506 to a light intensity which leads to rapid growth of the cyanobacterial biomass 502. Moreover, these steps are repeated to supply more light to the cyanobacterial biomass 502 as the biomass increases, until a cyanobacterial biomass 502 of 1g is calculated, as shown with respect to the density detection system 500.3.

The calculated cyanobacterial biomass 502 concentrations are measured using an application programming interface and subsequently one or more regulated growth conditions are customized based on the cyanobacterial biomass 502 concentration at a given stage of growth. Moreover, the density detection system 500 comprises coloured LED indicator on the base unit, for example, which is used to communicate one of three growing stages to the user: growing (orange light); growing but requires attention/tasks to be completed (orange flashing light); and ready to harvest (green). The ability for the density detection system 500 to do this is driven by the at least one photodiode and other PCB sensing systems, such as RGB sensors for example. Furthermore, detection of growth issues, such as discoloration or excessive culture foaming (a sign of poor culture health), relaying signal from the at least one photodiode and/or RGB sensors on the PCBs to a computing device as an alert for the problem.

Notably, the density detection system 500A shown in FIG. 5A measures the cyanobacterial biomass within a range of 0-1.5g/L, and the density detection system 500B shown in FIG. 5B measures the cyanobacterial biomass within a range of 1-3.5 g/L.

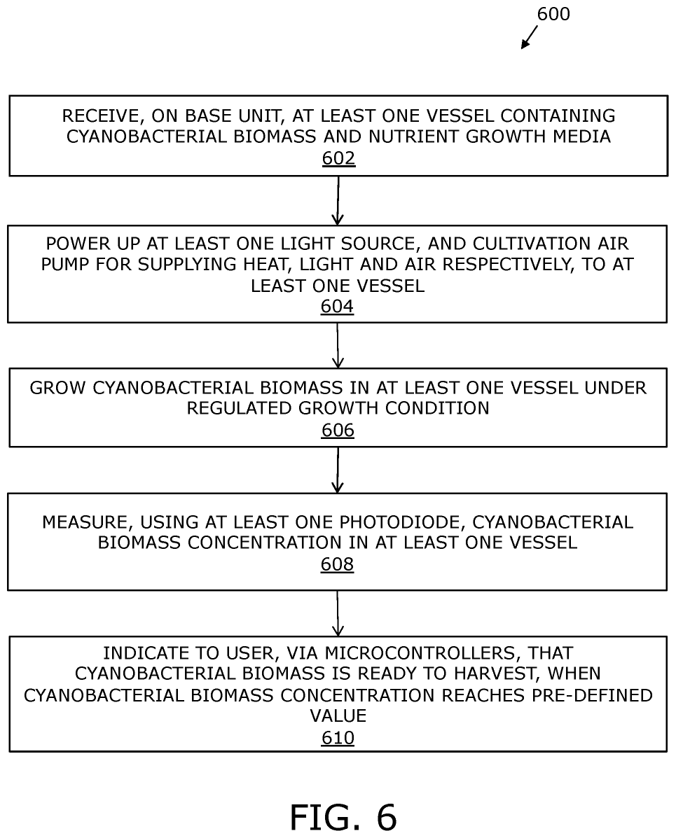

Referring to FIG. 6, illustrated is a flowchart 600 of steps of a method for (of) cultivating and harvesting a cyanobacterial biomass by using the aforementioned system, in accordance with an embodiment of the present disclosure. At step 602, at least one vessel containing the cyanobacterial biomass and the nutrient growth media is received on the base unit. At step 604, at least one light source, and a cultivation air pump is powered up for supplying heat, light and air respectively, to the at least one vessel. At step 606, the cyanobacterial biomass is grown in the at least one vessel under regulated growth conditions. At step 608, a cyanobacterial biomass concentration in the at least one vessel is measured using at least one photodiode. At step 610, when the cyanobacterial biomass concentration reaches a pre- defined value, the cyanobacterial biomass is ready to harvest is indicated to the user, via the microcontrollers.

The steps 602, 604, 606, 608 and 610 are only illustrative and other alternatives can also be provided where one or more steps are added, one or more steps are removed, or one or more steps are provided in a different sequence without departing from the scope of the claims herein.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural.

1. A system for cultivating and harvesting a cyanobacterial biomass, the system comprising:
at least one vessel that, when in operation, grows the cyanobacterial biomass in a nutrient growth media under regulated growth conditions, the at least one vessel having a top end, a bottom end, at least one side wall connecting the top end to the bottom end, and a one-way air valve at the bottom end;
at least one light source that, when in operation, supplies light and heat to the at least one vessel, wherein the at least one light source is arranged on a side of the at least one vessel;
at least one photodiode that, when in operation, measures a cyanobacterial biomass concentration in the at least one vessel, wherein the at least one photodiode is arranged opposite to the at least one light source;
a base unit configured to receive the bottom end of the at least one vessel; and
a cultivation air pump that, when in operation, supplies air to the at least one vessel via the one-way air valve therein.

2. A system of claim 1, further comprising
an electrical circuitry for supplying power to the at least one light source, the least one photodiode, and the cultivation air pump; and
microcontrollers configured to indicate, to a user, that the cyanobacterial biomass is ready to harvest, when the cyanobacterial biomass concentration reaches a pre-defined value.

3. A system of claim 1 or 2, wherein the at least one light source and the at least one photodiode is arranged on at least one printed circuit board, wherein the printed circuit board and microcontrollers are accommodated in a housing configured to partly cover the at least one vessel.

4. A system of any of the claim 1 or 2, wherein the cultivation air pump, the electrical circuitry, and microcontrollers are accommodated in the base unit.

5. A system of claim 1, further comprising a harvesting arrangement for harvesting cyanobacterial biomass, wherein the harvesting arrangement comprises
a double-ended harvesting vessel for receiving at least one set volume of spirulina culture; and
a threaded mesh component which can be attached to the harvesting vessel for separating the cyanobacterial biomass from the growth medium via filtration;
a mesh cap, to stop the leaking of liquid while the harvesting vessel is being shaken for the mixing of the harvested cyanobacterial biomass from the mesh into a liquid drink; and
a top cap for the harvesting vessel to stop the leaking of liquid while the harvesting unit is being shaken for the mixing of the harvested cyanobacterial biomass from the mesh into a liquid drink.

6. A system of any of the preceding claims, further comprising a software module executable on the microcontrollers, that is trained using machine learning algorithms or a model created from past cultivation data, to regulate growth conditions based on the cyanobacterial biomass concentration in the at least one vessel.

7. A system of any of the preceding claims, wherein the regulated growth conditions include:
growing the cyanobacterial biomass continuously;
a temperature in a range of 28 to 40° C.;
a photoperiod of 24 hours;
an illumination intensity in a range of 20 to 2500 μmol of photons/m²/s; and
a colour temperature in a range of 2000 to 7000K.

8. A system of any of the preceding claims, wherein one or more components of the system are fabricated using at least one of: an injection-moulding technique, a printing technique, an etching technique, or a weaving technique.

9. A system of any of the preceding claims, wherein the harvesting mesh has an operational area in a range of 50×50 mm to 150×150 mm.

10. An application programming interface executable on a computing device associated with a user of the system of any of claims 2-9, wherein the application programming interface is communicably coupled with the microcontrollers, and wherein the application programming interface is configured to provide a signal to the microcontrollers, for setting growth parameters that are used for regulating operation of the at least one light source, the at least one photodiode, the electrical circuitry, and the cultivation air pump.

11. A method for (of) cultivating and harvesting a cyanobacterial biomass by using a system of any of claims 2-10, the method comprising:
receiving, on the base unit, the at least one vessel containing the cyanobacterial biomass and the nutrient growth media;
powering up the at least one light source, and the cultivation air pump for supplying the light and air respectively, to the at least one vessel;
growing the cyanobacterial biomass in the at least one vessel under regulated growth conditions;
measuring, using the at least one photodiode, the cyanobacterial biomass concentration in the at least one vessel; and
indicating to the user, via the microcontrollers, that the cyanobacterial biomass is ready to harvest, when the cyanobacterial biomass concentration reaches the pre-defined value.

12. A method of claim 11, further comprising harvesting the cyanobacterial biomass, wherein harvesting comprises
pouring a set volume of spirulina culture from the cultivation vessel into the harvesting vessel with mesh component attached to separate cyanobacterial biomass from growth medium;
placing a mesh cap over a mesh component to seal the bottom of the harvesting vessel; and
collecting the cyanobacterial biomass by pouring fresh water into the harvesting vessel, attaching the top cap and shaking.

13. A method of claim 11 or 12, further comprising training a software module, executable on the microcontrollers, using machine learning algorithms, to regulate growth conditions based on the cyanobacterial biomass concentration in the at least one vessel.

14. A method of any of the claims 11 to 13, wherein the regulated growth conditions include:
growing the cyanobacterial biomass continuously;
a temperature in a range of 28° C. to 40° C.;
a photoperiod of 24 hours;
an illumination intensity in a range of 20 to 2500 μmol of photons/m²/s; and
a colour temperature in a range of 2000 to 7000K.

15. A method of any of the claims 11 to 14, wherein measuring the cyanobacterial biomass within a range of 0-1.5 g/L comprises:
a) arranging the at least one light source and a first photodiode on opposite sides of the at least one vessel;
b) illuminating the at least one vessel with the at least one light source, on one side of the at least one vessel, with a maximum light intensity;
c) measuring, using the first photodiode on the opposite side of the illuminated at least one light source, a unique value corresponding to the cyanobacterial biomass concentration at a given time;
d) determining, based on the unique value, a density of the cyanobacterial biomass at the given time; and
e) indicating to a user, via the microcontrollers, the density of the cyanobacterial biomass at the given time

16. A method of any of the claims 11 to 15, wherein measuring the cyanobacterial biomass within a range of 1-3.5 g/L comprises:
a) arranging a secondary light source opposite to a second photodiode on a printed circuit board;
b) illuminating within the at least one vessel with the secondary light source with a maximum light intensity;
c) measuring, using the second photodiode on the printed circuit board, a unique value corresponding to the cyanobacterial biomass concentration at a given time;
d) determining, based on the unique value, a density of the cyanobacterial biomass at the given time;
e) indicating to a user, via the microcontrollers, the density of the cyanobacterial biomass is equal to a harvesting density of the cyanobacterial biomass, wherein the harvesting density corresponds to a set value measured by the at least one photodiode.

17. A method of any of the claims 11 to 15, further comprising determining an optimum light intensity for a next stage of growth, the method comprising:
a) turning off the at least one light source;
b) illuminating the at least one vessel with the at least one light source with an incremental light intensity until a pre-defined photodiode value is measured using the at least one photodiode on the opposite side of the illuminated at least one light source;
c) determining a light intensity resulting in the pre-defined photodiode value as the optimum light intensity for the next stage of growth;
d) allowing the cyanobacterial biomass to grow for a pre-defined period with the optimum light intensity; and
e) repeating steps (a) to (d) until the density of the cyanobacterial biomass is equal to a harvesting density of the cyanobacterial biomass as obtained by step (f) of claim 15, wherein the harvesting density corresponds to a set value measured by the at least one photodiode.

18. A method of any of the claims 11 to 16, wherein the method comprises operating the microcontroller, based on a signal from an application programming interface communicably coupled with the microcontrollers, for regulating operation of the at least one light source, the at least one photodiode, the electrical circuitry, and the cultivation air pump.
